(19) United States
(12) Patent Application Publication
(10) Pub. No.: US 2021/0163289 A1
(43) Pub. Date: Jun. 3, 2021

(54) PROCESS FOR THE PRODUCTION OF A FERTILIZER

(71) Applicant: NOURYON CHEMICALS INTERNATIONAL B.V., ARNHEM (NL)

(72) Inventors: Martinus Catharinus TAMMER, Diepenveen (NL); Michel VAN DEN BERG, Elst (NL); Bjorn TER HORST, Maarn (NL)

(73) Assignee: NOURYON CHEMICALS INTERNATIONAL B.V., ARNHEM (NL)

(21) Appl. No.: 16/772,838

(22) PCT Filed: Jan. 22, 2019
(86) PCT No.: PCT/EP2019/051512

§ 371 Date: Jun. 15, 2020

(30) Foreign Application Priority Data
Jan. 25, 2018 (EP) 18153427.2

(51) Int. Cl. C01B 25/30; C07C 51/60

(52) U.S. Cl. CPC C01B 25/303; C07C 51/60

(57) ABSTRACT

Process for the production of a compound comprising potassium phosphite comprising the steps of (a) reacting carboxylic acid of the formula R—(C(═O)OH)ₙ with phosphorous trichloride (PCl₃) towards a mixture comprising phosphorous acid (H₃PO₃) and acid chloride of the formula R—(C(═O)Cl)ₙ; wherein R is a linear or branched alkyl or alkanediyl group with 1-20 carbon atoms and n is 1 or 2, (b) subjecting said mixture to a separation step, thereby obtaining (i) a fraction comprising crude phosphorous acid (H₃PO₃) and (ii) a fraction comprising acid chloride, (c) combining water, a potassium compound selected from KOH, KHCO₃ and K₂CO₃, and the fraction comprising crude phosphorous acid, thereby forming an aqueous solution comprising potassium phosphite, and (d) removing organic compounds from said aqueous solution.

### CROSS-REFERENCE TO RELATED APPLICATION

This application is a U.S. National-Stage entry under 35 U.S.C. § 371 based on International Application No. PCT/EP2019/051512, filed Jan. 22, 2019, which was published under PCT Article 21(2) and which claims priority to European Application No. 18153427.2, filed Jan. 25, 2018, which are all hereby incorporated in their entirety by reference.

### TECHNICAL FIELD

The present invention relates to a process for the removal of organics from an acid chloride production waste stream. The invention also relates to the production of potassium phosphite.

### BACKGROUND

Acid chlorides are conventionally produced by reacting a carboxylic acid with PCl₃ to form acid chloride and phosphorous acid (H₃PO₃). This H₃PO₃ is a waste stream that cannot be sent to a biological waste water treatment unit, as its phosphorous content is too high. It is therefore desired to use this material in another process.

Such use, however, requires that organics (e.g. acid chloride residues) are removed from the crude H₃PO₃. Unfortunately, removal of organics from crude H₃PO₃ is difficult. Even after removal of an organic layer, the H₃PO₃ remains unstable: new organic layers keep forming during storage, finally resulting in blackening of the H₃PO₃. In addition, other objects, desirable features and characteristics will become apparent from the subsequent summary and detailed description, and the appended claims, taken in conjunction with the accompanying drawings and this background.

### SUMMARY

An object of the present invention is therefore the provision of a process that enables quick and easy removal of organics from H₃PO₃-containing waste streams.

It has now been found that this object can be achieved by reacting the crude H₃PO₃ with a potassium compound towards potassium phosphite. Organics can be easily removed from the resulting potassium phosphite solution, resulting in a clear potassium phosphite solution.

Potassium phosphite is used in the agro industry as an antifungal fertilizer. It is a fungicide and at the same time contains an important nutrient: potassium. It is non-toxic and provides both protective and curative responses against various fungal pathogens, like Phytophtora, Rhizoctonia, Pythium, and Fusarium.

For its application as fungicide and/or fertilizer, potassium phosphite is generally sold in aqueous solutions of about 50 wt %, and used in strong dilution.

Transforming crude H₃PO₃ waste streams into potassium phosphite not only enables quick and easy removal of organics from the acid chloride waste stream, it also allows for the production of a valuable product from a waste stream and the production of potassium phosphite from a sustainable source.

An object of the present invention is therefore the provision of a process that enables quick and easy removal of organics from H₃PO₃-containing waste streams. Another object is the production of potassium phosphite from waste phosphorous acid. A further object is to turn waste phosphorous acid into high grade material.

It has now been found that this object can be achieved by reacting the crude H₃PO₃ with a potassium compound towards potassium phosphite. Organics can be easily removed from the resulting potassium phosphite solution, resulting in a clear potassium phosphite solution.

### DESCRIPTION

The following detailed description is merely exemplary in nature and is not intended to limit the invention or the application and uses of the invention. Furthermore, there is no intention to be bound by any theory presented in the preceding background of the invention or the following detailed description.

The present invention relates to a process for the production of a compound comprising potassium phosphite, comprising the steps of:

a. reacting carboxylic acid of the formula R—(C(═O)OH)ₙ with phosphorous trichloride (PCl₃) towards a mixture comprising phosphorous acid (H₃PO₃) and acid chloride of the formula R—(C(═O)CL)ₙ; wherein R is a linear or branched alkyl or alkanediyl group with 1-20 carbon atoms and n is 1 or 2,
b. subjecting said mixture to a separation step, thereby obtaining (i) a fraction comprising crude phosphorous acid (H₃PO₃) and (ii) a fraction comprising acid chloride,
c. adding water and a potassium compound selected from KOH, KHCO₃ and K₂CO₃ to the fraction comprising crude phosphorous acid, thereby forming an aqueous solution comprising potassium phosphite, and
d. removing organic compounds from said aqueous solution.

The resulting aqueous solution comprises potassium phosphite. The term potassium phosphite refers to compounds with the general formula KₓH₃₋ₓPO₃—wherein x is an average value in the range 1-2—such as K₂HPO₃, KH₂PO₃, and combinations thereof.

Potassium phosphite can be used as a fungicide and/or fertilizer in the agro industry. The resulting acid chloride can be used for the synthesis of other organic compounds, such as organic peroxides, more specifically diacyl peroxides and peroxyesters.

### Step a

This step involves the reaction between a carboxylic acid and PCl₃.

The carboxylic acid has the formula R—(C(═O)OH)n, wherein R is a linear or branched alkyl or alkanediyl group with 1-20, preferably 3-17, even more preferably 5-17, and most preferably 7-17 carbon atoms. The value of n is either 1 (resulting in a mono-acid) or 2 (resulting in a di-acid).

Examples of preferred carboxylic acids are mono-acids. Preferred monoacids are isobutanoic acid, n-butanoic acid, neopentanoic acid (pivalic acid), n-pentanoic acid (valeric acid), n-hexanoic acid, n-octanoic acid, 2-ethylhexanoic acid, 3,5,5-trimethylhexanoic acid, (neo)heptanoic acid, (neo)decanoic acid, cyclohexane carboxylic acid, and lauric acid.

The carboxylic acid functionalities and the PCl3 react in a molar ratio 3:1, but it is preferred to use an excess of PCl3. Preferably, a molar excess of 0-80%, more preferably of 10-50%, and most preferably of 15-40% PCl3 is used.

The carboxylic acid functionalities and the PCl3 are therefore preferably reacted in a molar ratio of carboxylic acid functionalities to PCl3 of 1.5:1-3.0:1, more preferably 2.0:1-2.7:1, and most preferably 2.2:1-2.6:1. Hence, if a mono-acid is used, it is preferably reacted in a molar ratio carboxylic acid to PCl3 of 1.5:1-3.0:1, more preferably 2.0:1-2.7:1, and most preferably 2.2:1-2.6:1. If a di-acid is used, it is preferably reacted in a molar ratio carboxylic acid to PCl3 of 0.75:1-1.5:1, more preferably 1.0:1-1.35:1, and most preferably 1.1:1-1.3:1.

The reaction is preferably performed at a temperature in the range 20-80° C., more preferably 30-70° C., and most preferably 40-65° C.

The reaction is preferably conducted in the absence of water and organic solvents.

In a preferred embodiment, the reaction is conducted in an oxygen-free or oxygen-lean atmosphere.

The reaction results in the formation of an acid chloride and phosphorous acid.

The acid chloride has the formula R—(C(═O)Cl)n, wherein R is a linear or branched alkyl group with 1-20, preferably 3-17, even more preferably 5-17, and most preferably 7-17 carbon atoms and n is either 1 or 2. If n is 1, the acid chloride is a mono-acid chloride; if n is 2, the acid chloride is a di-acid chloride.

Examples of preferred acid chlorides are mono-acid chlorides. Preferred mono-acid chlorides are isobutyryl chloride, n-butyryl chloride, neopentanoyl chloride (pivaloyl cloride), n-pentanoyl chloride (valeroyl chloride), hexanoyl chloride, n-octanoyl chloride, 2-ethylhexanoyl chloride, 3,5,5-trimethylhexanoyl chloride, (neo)heptanoyl chloride, (neo)decanoyl chloride, cyclohexane carbonyl chloride, and lauroyl chloride.

By-products may be formed in this step, such as HCl, the anhydride of the carboxylic acid, and anhydrides of the carboxylic acid and phosphorous acid. HCl and any other exiting fumes can be led through a scrubber.

### Step b

The reaction product of step a) is a bi-phasic mixture comprising a H₃PO₃-containing phase (the crude phosphorous acid-comprising fraction) and an organic phase comprising the acid chloride (the acid chloride-comprising fraction). In step b), these two phases are separated. Separation can be conducted in any suitable way, e.g. by gravity or centrifugation.

The resulting separated crude phosphorous acid-comprising fraction contains phosphorous acid and organic contaminants. It may also contain a small amount of PCl3. The total organic contaminant concentration is generally not higher than 5.0 wt %, more preferably not higher than 2.0 wt %, and most preferably not higher than 1.0 wt %.

The acid chloride can be used as a reactant towards various chemicals. Mono-acid chlorides can be used as reactants towards various esters, anhydrides, amides, and organic peroxides, in particular diacyl peroxides and peroxyesters. Di-acid chlorides can be used to produce polyesters and polyamides.

In order to make diacyl peroxides, the mono-acid chloride is reacted with hydrogen peroxide and an alkali metal salt (or a reaction product thereof) to form symmetrical diacyl peroxides. In order to prepare asymmetrical diacyl peroxides, the acid chloride can be reacted with a peroxyacid. Peroxyesters are prepared by reacting the acid chloride with an organic hydroperoxide. These processes are well known in the art.

Preferred organic peroxides to be prepared from the acid chloride resulting from the present invention are di-isobutyryl peroxide, di-n-butyryl peroxide, di-neopentanoyl peroxide (di-pivaloyl peroxide), di-n-pentanoyl peroxide (di-valeroyl peroxide), di-hexanoyl peroxide, di-1-octanoyl peroxide, di-2-ethylhexoyl peroxide, di-1-nonanoyl peroxide, di-3,5,5-trimethylnonanoyl peroxide, di-neodecanoyl peroxide, and di-lauroyl peroxide.

### Step c

The crude phosphorous acid-comprising fraction is then reacted with a potassium compound selected from KOH, KHCO₃ and K₂CO₃, thereby forming an aqueous KH₂PO₃ solution. KOH is the preferred potassium compound, since the use of K₂CO₃ and KHCO₃ will lead to CO₂ production.

Water, potassium compound, and the crude phosphorous acid fraction can be combined in any order, as long as the mixture is sufficiently cooled to control the resulting exothermal reaction.

Hence, water and potassium compound can be pre-mixed and the crude phosphorous acid fraction can be dosed to said mixture. Alternatively, some of the water can be added to the crude phosphorous acid fraction or vice versa, followed by dosing an aqueous solution of the potassium compound. Alternatively, an aqueous solution of the potassium compound can be added to the crude phosphorous acid fraction or vice versa, followed by dosing the remaining amount of water.

In a preferred embodiment, water and crude phosphorous acid fraction are combined at such a rate that any PCl3 that is present in the crude phosphorous acid fraction reacts to form pure, undiluted HCl (which evaporates as a result of the exothermal reaction).

The amounts of water and potassium compound that are combined with the crude phosphorous acid fraction are preferably such that the pH of the resulting solution remains below 5, more preferably below 4.5. If the pH exceeds this value, extraction of organics in step d) will become difficult, because water soluble K-salts of the acid chlorides will be formed.

Phosphorous acid and potassium compound are preferably combined in a molar ratio of around 1:1. It is not desired to use a significant excess of one of the components. Excess of either will decrease or increase the pH of the final solution.

The product resulting from this step is an aqueous potassium phosphite solution. The potassium phosphite concentration in this solution is preferably at least 20 wt %, more preferably at least 30 wt %, and most preferably at least 40 wt %. The potassium phosphite concentration is preferably at most 70 wt %, more preferably at most 60 wt %, and most preferably at most 50 wt %.

During the process, small amounts/traces of KH₂PO₄ may be formed. The amount KH₂PO₄ in the final solution is preferably below 10 wt %, more preferably below 5 wt % and most preferably below 1 wt %, based on the weight of potassium phosphite.

During the reaction, a precipitate may be formed, consisting or organic compounds and resulting from the organic contaminants in the phosphorous acid solution.

### Step d

In this step, the organics are removed from the solution. Depending on whether the organics are in solid or liquid form, this step can be conducted by filtration, centrifugation, distillation, steam distillation, stripping with air or nitrogen, or extraction. This step can be conducted before the addition of the potassium compound (i.e. before step c) and/or after the formation of potassium phosphite (i.e. after step c).

Examples of extraction agents are C5-20 alkanes (such as pentane, hexane, heptane, octane, nonane, decane, undecane, dodecane, tridecane, tetradecane, pentadecane, hexadecane, heptadecane, nonadecane, icosane, toluene, xylene, cyclohexane and their isomers and mixtures of such alkanes) and organic esters such as natural oils and esters of C1-18 mono-, di-, tri-, tetra-, or poly-alcohols (preferably ethanol, propanol, butanol, glycerol) and C2-24 mono-, di-, tri-, tetra-, or poly-acids (preferably benzoic acid, phthalic acid, 1,2-cyclohexane dicarboxylic acid, capric acid, lauric acid, myristic acid, palmitic acid, stearic acid and oleic acid).

Preferred extraction agents are materials that have food or food contact approval, such as di-isononyl-1,2-cyclohexaandicarboxylate (DINCH) and natural oils having such approval.

The resulting aqueous solution can be used—after further dilution—as a fungicide and/or fertilizer.

### EXAMPLES

### Example 1

### Steps a) and b)

Dodecanoic acid (518 g, 2.59 mol) was charged to a three-necked round bottom flask with bottom drain equipped with a mechanical overhead stirrer, a thermometer, a reflux cooler, a dropping funnel and a nitrogen purge. The reflux cooler was connected to a double wash vessel to trap HCl and PCl3 (first flask was empty; the second one was filled with water).

Dodecanoic acid was heated to 63° C., resulting in a transparent melt. PCl3 (100 ml, 1.15 mol) was added via the dropping funnel in 20 to 30 minutes; the temperature was maintained at 63° C. Stirring was stopped after the addition of the PCl3 and the mixture was left to stand for 3 hours at 63° C. The warm crude H₃PO₃ solution (containing PCl3 and several polyphosphorous compounds) was collected at the bottom of the flask and this phase was drained off as a slightly hazy and viscous liquid (73 g, 890 mmol, 78% yield). The remaining crude dodecanoyl chloride (518 g, 2586 mmol, 102%) was isolated as a colorless hazy liquid and was used as such in the production of dilauroyl peroxide.

### Steps c) and d)

Water (13.32 g) and KOH (50.0 g, 45 wt %, 0.40 mol) were charged to a 100 mL beaker, equipped with a bottom drain and thermometer. The solution was stirred with a mechanical overhead stirrer at 600 rpm. The beaker was placed in a 1000 mL beaker containing ice water and was cooled down to 5° C. 47 gram of the crude H₃PO₃ solution of step b) was added to the stirred solution. The dosing speed was set at such a rate that the temperature never exceeded 30° C. (dosing took 20 minutes). During said dosing, a white precipitate was formed. After the dosing, stirring was stopped and the reaction mixture was cooled to 10° C. The chilled mixture was filtered under reduced pressure over a G3 glass filter. The resulting slightly hazy solution was subsequently filtered over a G4 glass filter. The second filtration yielded a clear colorless 50 wt % KH₂PO₃ solution (88.2 g, 92% yield). Total organics <0.1 wt % (by NMR); total chloride content 0.36 wt %.

### Example 2

Steps a) and b) of Example 1 were repeated.

Water (16.5 g) was carefully added to stirred crude H₃PO₃ (38.4 g, 0.47 mol). The temperature of the mixture rose quickly, which resulted in HCl formation (the crude H₃PO₃ contained some PCl3 residues). The vapours were removed by a nitrogen purge. When the addition was completed, the resulting hazy, hot 70 wt % H₃PO₃ solution was left to cool to room temperature. During cooling, an organic phase accumulated on the top of the solution. The phases were separated by draining off the aqueous H₃PO₃-containing phase (bottom layer).

47 gram of the so-obtained 70 wt % H₃PO₃ solution (0.40 mol H₃PO₃) was charged to a 100 mL beaker, equipped with a bottom drain and thermometer. The solution was stirred with a mechanical overhead stirrer at 1400 rpm. The beaker was placed in a 1000 mL beaker containing ice water and was cooled down to 5° C. A 45 wt % KOH solution (50 gram, 0.40 mol KOH) was added in dropwise fashion via a dropping funnel at such a rate that the temperature was kept below 25° C. The addition took 24 minutes. After this addition, stirring was stopped and a slightly hazy solution with floating solids on top was observed. The slightly hazy solution was drained off and the solids were left behind in the beaker. The obtained KH₂PO₃ solution (50 wt %, density=1.38 g/ml) was subsequently filtered over a G4 glass filter, resulting in a clear and colorless solution (93.94 gram KH₂PO₃).

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1.-12. (canceled)

13. A process for producing a compound comprising potassium phosphite of formula KₓH₃₋ₓPO₃ wherein x is an average value in the range 1-2, the method comprising the steps of:
a. reacting carboxylic acid of the formula R—(C(═O)OH)ₙ with phosphorous trichloride (PCl₃) to make a mixture comprising phosphorous acid (H₃PO₃) and acid chloride of the formula R—(C(═O)Cl)ₙ; wherein R is a linear or branched alkyl or alkanediyl group with 1-20 carbon atoms and n is 1 or 2,
b. subjecting said mixture to a separation step, thereby obtaining (i) a fraction comprising crude phosphorous acid (H₃PO₃) and (ii) a fraction comprising acid chloride, and
c. combining water, a potassium compound selected from KOH, KHCO₃ and K₂CO₃, and the fraction comprising crude phosphorous acid, thereby forming an aqueous solution comprising potassium phosphite, and
d. removing organic compounds from said aqueous solution.

14. The process according to claim 13, wherein in step c) the fraction comprising crude phosphorous acid is dosed to an aqueous solution of the potassium compound.

15. The process according to claim 13, wherein step d) is performed by centrifugation, filtration, extraction, distillation, steam distillation, or stripping with air or nitrogen.

16. The process according to claim 13, wherein R is a linear or branched alkyl or alkanediyl group with 3-17 carbon atoms.

17. The process according to claim 16, wherein R has 7-17 carbon atoms.

18. The process according to claim 13, wherein the carboxylic acid comprises isobutanoic acid, n-butanoic acid, lauric acid, 3,5,5-trimethylhexanoic acid, pivaloic acid, valeric acid, n-hexanoic acid, n-octanoic acid, 2-ethylhexanoic acid, (neo)decanoic acid, (neo)heptanoic acid, or cyclohexane carboxylic acid.

19. The process according to claim 13, wherein the potassium compound is KOH.

20. The process according claim 13, wherein n is 1.

21. The process according to claim 13, comprising the additional step of reacting the acid chloride with hydrogen peroxide and an alkali metal salt to form a diacyl peroxide.

22. The process according to claim 13, comprising the additional step of reacting the acid chloride with a peroxyacid and an alkali metal salt to form a diacyl peroxide.

23. The process according to claim 22, wherein the peroxyacid comprises peracetic acid, perpropionic acid, or perlauric acid.

24. Process according to claim 13, comprising the additional step of reacting the acid chloride with an organic hydroperoxide and an alkali metal salt to form a peroxyester.

25. The process according to claim 24, wherein the organic hydroperoxide comprises tert-butyl hydroperoxide, tert-amyl hydroperoxide, cumyl hydroperoxide, 2,2,4,4-tetramethylbutyl hydroperoxide, 1,3-bis(2-hydroperoxypropan-2-yl)benzene, 1,4-bis(2-hydroperoxypropan-2-yl)benzene, 2,5-dihydroperoxy-2,5-dimethylhexane, 2,5-dihydroperoxy-2,5-dimethylhex-3-yne, and mixtures thereof.

26. The process according to claim 13, further comprising the step of
d. removing organic compounds from the aqueous solution.

27. The process according to claim 26, further comprising the step of reacting the acid chloride with hydrogen peroxide, a peroxy acid, or an organic hydroperoxide.

28. The process according to claim 26, wherein R has 7-17 carbon atoms, n is 1, and the potassium compound is KOH.

29. The process according to claim 26, wherein the potassium phosphite comprises KH₂PO₃.

30. The process according to claim 26, wherein the potassium phosphite comprises K₂HPO₃.

31. The process according to claim 26, wherein n is 2
